(19) United States
(12) Patent Application Publication
(10) Pub. No.: US 2025/0122480 A1
(43) Pub. Date: Apr. 17, 2025

(54) METHOD FOR PREPARING AUTOLOGOUS SKIN CELL SUSPENSION

(71) Applicants: Peking Union Medical College Hospital, Chinese Academy of Medical Sciences, Beijing (CN); Beijing Vistek Medical Equipment Co., Ltd, Beijing (CN); Jiangxi Boshen Medical Device Co., Ltd, Fuzhou (CN)

(72) Inventors: Zhifei Liu, Beijing (CN); Hongsheng Li, Beijing (CN); Jianshan Wu, Fuzhou (CN); Wenchao Zhang, Beijing (CN)

(21) Appl. No.: 18/824,945

(22) Filed: Sep. 5, 2024

(30) Foreign Application Priority Data
Oct. 11, 2023 (CN) 202311314999.2

(51) Int. Cl. C12N 5/071

(52) U.S. Cl. CPC C12N 5/0698; C12N 2509/00

(57) ABSTRACT

A method for preparing autologous skin cell suspension includes at least one of the following steps: scraping a digested skin tissue by using a first cell separation unit to obtain a first skin cell suspension and a skin tissue residue; and transferring the skin tissue residue to a second cell separation unit through a conduit groove defined on the first cell separation unit to prepare a second skin cell suspension; and preparing the digested skin tissue by using the second cell separation unit to obtain a third skin cell suspension. Specially, the digested skin tissue is directly placed on a filter screen, and a grinding rod that matches a shape of the filter screen is used to grind the digested skin tissue with slight adding pressure.

### CROSS-REFERENCE TO RELATED APPLICATION

This application claims priority to a Chinese patent application No. 202311314999.2, filed to China National Intellectual Property Administration (CNIPA) on Oct. 11, 2023, which is herein incorporated by reference in its entirety.

### TECHNICAL FIELD

The present disclosure relates to the field of medical equipment, and more particularly to a method for preparing autologous skin cell suspension (ASCS).

### BACKGROUND

Autologous active epidermal cell transplantation and regeneration technology is a technical solution widely used for treatment of wounds, burns, scars, and skin defects. This technology utilizes regenerative ability of human body's own skin, which specially uses an autologous healthy skin sample to prepare a skin cell suspension to promote skin growth. The prepared skin cell suspension is directly applied or sprayed onto the skin wounds treated with a micro-dynamic system, which is used for treatments of stable-phase vitiligo, post-acne scars, and superficial scars.

At present, there are two methods for preparing skin cells, i.e., a mechanical preparation method and a digestive enzyme preparation method. The mechanical preparation method is to obtain skin cells by directly grinding, centrifuging, etc., small pieces of skin tissue. However, the mechanical preparation method is cumbersome in preparation process, poor in effect, and causes damage to the skin cells. The digestive enzyme preparation method involves treating the skin tissue with a digestive enzyme, and scraping the skin tissue with a scalpel to obtain the skin cells. The scraping operation performed by the scalpel has high requirements on the skin sample. Specially, a relatively large, smooth, and complete skin sample is easy to operate; otherwise, a small and irregular skin sample is difficult to operate. In addition, during scraping the skin cells, a large number of cell aggregates and residual tissue fragments are formed. Furthermore, after filtering the scrapped skin cells, there are many residual tissues, and a large number of skin cells are not separated.

### SUMMARY

An objective of the present disclosure is to provide a method for preparing autologous skin cell suspension (ASCS), which can separate the skin cells more completely and quickly, and improve separation efficiency of the skin cells.

In order to achieve the above objective, the present disclosure provides the method for preparing ASCS. Specially, the skin tissue that has been digested by the enzyme is directly placed on a filter screen, and a grinding tool (i.e., grinding rod) that matches the shape of the filter screen is used to grind the skin tissue with slight adding pressure. The pressure generated by the grinding rod interacts with a shear force formed between meshes of the filter screen and the grinding rod, which is equivalent to a matrix effect of countless dense blades. Therefore, the speed of scraping the skin cells in the present disclosure is greatly improved compared to using a single blade of the regular scalpel. Moreover, the large number of cell aggregates and residual tissue fragments formed in scraping the skin sample with the single blade of a regular scalpel are avoided. Therefore, the separation efficiency of skin cells is also greatly improved.

The method is applied in a preparation device for the ASCS, which includes a digestion unit, a digestive enzyme washing unit, a first cell separation unit, and a second cell separation unit. The first cell separation unit defines a conduit groove, the second cell separation unit defines an opening, and the conduit groove defined on the first cell separation unit is disposed above the opening defined on the second cell separation unit.

The method for preparing the ASCS includes at least one of the following steps: scraping a digested skin tissue by using the first cell separation unit to obtain a first skin cell suspension and a skin tissue residue; and transferring the skin tissue residue to the second cell separation unit through the conduit groove defined on the first cell separation unit to prepare a second skin cell suspension; and preparing the digested skin tissue by using the second cell separation unit to obtain a third skin cell suspension.

Compared with the related art, in the method for preparing the ASCS provided by the present disclosure, when the digested skin tissue is prepared into a large piece, the first cell separation unit can be first used to scrape off the digested skin tissue to obtain the first skin cell suspension and the skin tissue residue. Then, the skin tissue residue is transferred to the second cell separation unit through the conduit groove defined on the first cell separation unit for re-preparation to obtain the second skin cell suspension. When the digested skin tissue is prepared into a small piece, the digested skin tissue can be directly placed on the second cell separation unit and prepared to obtain the third skin cell suspension. Namely, when the digested skin tissue is prepared into the large piece, the digested skin tissue can be directly separated by using the first cell separation unit to obtain the skin cell suspension, or the digested skin tissue can be separated by using the first cell separation unit to obtain the skin cell suspension, and then the skin tissue residue is transferred to the second cell separation unit for secondary separation, so that the skin cells can be separated more completely to obtain the skin cell suspension as much as possible. When the digested skin tissue is prepared into the small piece, the second cell separation unit can be directly used to obtain the skin cell suspension. Therefore, no matter whether the digested skin tissue is large piece or small piece, the digested skin tissue can be prepared by the first cell separation unit and the second cell separation unit, so that the skin cells are separated more completely, and the separation efficiency of the skin cells is improved.

It can be seen from the above that the method for preparing the ASCS provided by the present disclosure enables the separation of skin cells to be more complete and improves the separation efficiency of the skin cells.

### BRIEF DESCRIPTION OF DRAWINGS

Attached drawings described herein are used to provide a further understanding of the present disclosure and constitute a part of the present disclosure. However, illustrated embodiments of the present disclosure and the description thereof are used to explain the present disclosure, and do not constitute an improper limitation on the present disclosure.

FIG. 1 illustrates a schematic structural diagram of a preparation device for ASCS.

FIG. 2 illustrates a schematic structural diagram of a first cell separation unit according to an embodiment of the present disclosure.

FIG. 3 illustrates a schematic top view of the first cell separation unit according to the embodiment of the present disclosure.

FIG. 4 illustrates a schematic front view of the first cell separation unit according to the embodiment of the present disclosure.

FIG. 5 illustrates a schematic structural diagram of a filter screen according to an embodiment of the present disclosure.

FIG. 6 illustrates a schematic structural diagram of a grinding rod according to the embodiment of the present disclosure.

FIG. 7 illustrates a schematic structural diagram of a filter screen according to another embodiment of the present disclosure.

FIG. 8 illustrates a schematic structural diagram of a grinding rod according to another embodiment of the present disclosure.

FIG. 9 illustrates a method for preparing ASCS according to an embodiment of the present disclosure.

FIG. 10 illustrates a flowchart for preparing to-be-digested skin tissue according to an embodiment of the present disclosure.

Description of reference signs are as follows:

100—digestion unit; 200—digestive enzyme washing unit; 210—opening; 300—first cell separation unit; 310—operation plane; 320—incline plane; 330—conduit groove; 340—cell separation operation table; 400—second cell separation unit; 410—opening; 420—filter screen; 430—grinding rod; 440—preparation container; 500—display screen; 600—functional panel.

### DETAILED DESCRIPTION OF EMBODIMENTS

In order to make the technical problems to be solved, the technical solutions, and beneficial effects of the present disclosure clearer, the present disclosure will be further described in detail below with reference to the attached drawings and embodiments. It should be understood that the illustrated embodiments described herein are only used to explain the present disclosure and are not intended to limit the present disclosure.

In addition, the terms “first” and “second” are used for descriptive purposes only and are not to be construed as indicating or implying relative importance or implicitly indicating the number of indicated technical features. Thus, features defined with “first” and “second” may explicitly or implicitly include one or more of the features. In the description of the present disclosure, “a plurality of” means two or more, unless otherwise specifically defined. The meaning of “several” is one or more, unless specifically defined otherwise.

At present, there are two methods for preparing skin cells, i.e., a mechanical preparation method and a digestive enzyme preparation method. The mechanical preparation method is to obtain skin cells by directly grinding, centrifuging, etc., small pieces of skin tissue to obtain the skin cells. However, the mechanical preparation method is cumbersome in preparation process, poor in effect, and causes damage to the skin cells. The digestive enzyme preparation method involves treating the skin tissue with a digestive enzyme, and scraping the skin tissue with a scalpel to obtain the skin cells. The scraping operation performed by the scalpel has high requirements on skin sample. Specially, a relatively large, smooth, and complete skin sample is easy to operate; otherwise a small and irregular skin sample is difficult to operate. In addition, after filtering the scrapped skin cells, there are many residual tissues, and a large number of skin cells are not separated.

In view of the above technical problems, an embodiment of the present disclosure provides a method for preparing ASCS, which solves the problem of incomplete separation of skin cells in the preparation process of the skin cell suspension in the related art. Therefore, the separation of skin cells in the preparation process of the skin cell suspension provided by the embodiment of the present disclosure is more complete, and the separation efficiency of the skin cells in the present disclosure is improved. It should be understood that the method for preparing the ASCS is applied in a preparation device for the ASCS, which includes a digestion unit, a digestive enzyme washing unit, a first cell separation unit, and a second cell separation unit.

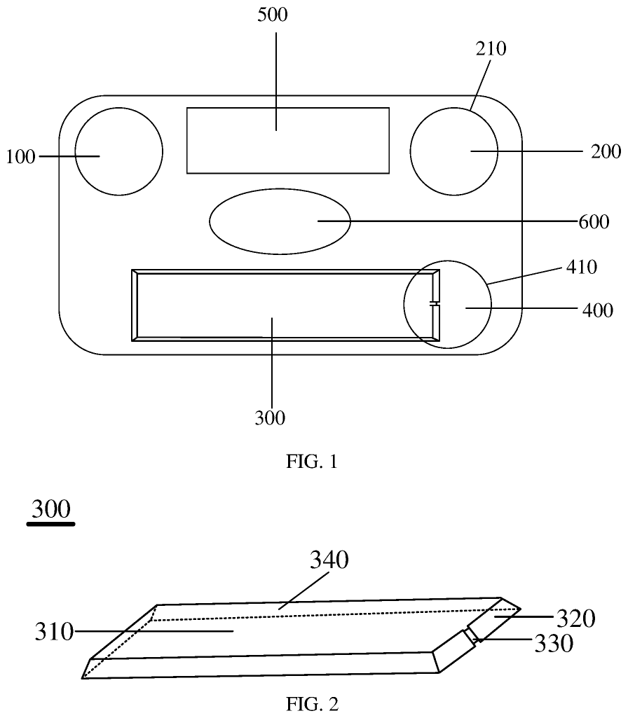

FIG. 1 shows a schematic structural diagram of the preparation device for the ASCS according to an embodiment of the present disclosure. As shown in FIG. 1, the preparation device for the ASCS according to the embodiment of the present disclosure includes: a digestion unit 100, a digestive enzyme washing unit 200, a first cell separation unit 300, and a second cell separation unit 400. Specially, the first cell separation unit 300 defines a conduit groove 330, and the conduit groove 330 is placed above an opening 410 defined on the second cell separation unit 400. It should be understood that the digestion unit 100 can be a digestion tank that accommodates a digestive enzyme solution therein for digesting the skin tissue (i.e., to-be-digested skin tissue). The digestive enzyme washing unit 200 can be a digestive enzyme washing tank that accommodates a digestive enzyme termination solution therein for terminating the action of the digestive enzyme and washing off the remaining digestive enzyme on the skin tissue (i.e., digestive enzyme-contained digested skin tissue). The first cell separation unit 300 can be a skin cell scraping and separating device by using a scalpel. The second cell separation unit 400 can be a suspension tank, a bottom of the suspension tank faces downwards to form a shallow bottom, a shape of the suspension tank is a cylinder, the opening 410 defined on the second cell separation unit 400 is 1 centimeter (cm) lower than that of the digestion tank and the digestive enzyme washing tank, and the second cell separation unit 400 is used for preparing the cell suspension.

It can be understood that the first cell separation unit 300 defines the conduit groove 330, and the conduit groove 330 is disposed above the opening 410 of the second cell separation unit 400. Namely, the first cell separation unit 300 is actually partially overlapped on the opening 410 of the second cell separation unit 400, thereby facilitating transferring the scraped skin cell suspension and the skin tissue residue in the first cell separation unit 300 into the second cell separation unit 400.

In an illustrated embodiment, when the digested skin tissue is the large piece, an incline plane 320 of the first cell separation unit can be first used to scrape off the digested skin tissue to obtain the first skin cell suspension and the skin tissue residue. Then, the skin tissue residue is transferred to the second cell separation unit through the conduit groove 330 defined on the first cell separation unit for re-preparation to obtain the second skin cell suspension. When the digested skin tissue is the small piece, the digested skin tissue can be directly placed on the second cell separation unit to obtain the third skin cell suspension. Namely, when the digested skin tissue is the large piece, the digested skin tissue can be first separated by using the first cell separation unit to obtain the skin cell suspension, and then the skin tissue residue is transferred to the second cell separation unit for secondary separation, so that the skin cells can be separated more completely to obtain the skin cell suspension as much as possible. When the digested skin tissue is the small piece, the second cell separation unit can be directly used to obtain the skin cell suspension. Therefore, no matter whether the digested skin tissue is the large piece or the small piece, the digested skin tissue can be prepared by the first cell separation unit and the second cell separation unit, so that the skin cells are separated more completely, and the separation efficiency of the skin cells is improved.

It should be noted that “first”, “second”, and “third” are merely used to distinguish the skin cell suspension obtained in different steps, and are not used to limit the type, quantity, or other features of the skin cell suspension. Namely, the first skin cell suspension merely refers to a cell suspension obtained by using the first cell separation unit to scrape the digested skin tissue of large patches, the second skin cell suspension merely refers to a cell suspension obtained by using the second cell separation unit, and the third cell suspension merely refers to a cell suspension obtained by directly separating the digested skin tissue of small patches by using the second cell separation unit.

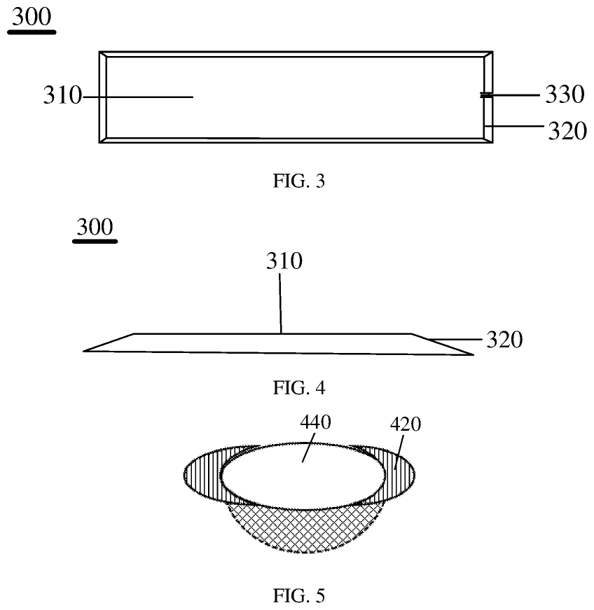

In an embodiment, FIG. 2 shows a schematic structural diagram of the first cell separation unit according to an embodiment of the present disclosure. FIG. 3 shows a schematic top view of the first cell separation unit according to the embodiment of the present disclosure. FIG. 4 shows a schematic front view of the first cell separation unit according to the embodiment of the present disclosure. As shown in FIGS. 2-4, the first cell separation unit 300 according to the embodiment of the present disclosure further includes a cell separation operation table. Specially, the cell separation operation table includes an operation plane 310, an incline plane 320 extending outwards from the operation plane 310, a conduit groove 330 defined on the incline plane 320, and the first cell separation unit 300 is connected to the opening defined on the second cell separation unit 400 through the conduit groove 330.

It should be understood that the first cell separation unit 300 can be in a form of a tray, a rectangle, or a semicircle, which is not limited herein. The embodiment of the present disclosure is specifically explained by taking a rectangular tray as an example. The first cell separation unit 300 can further include a cell scraping device. Specially, the cell scraping device can be a scalpel, which can be used for manual operation to achieve the cell scraping and separation. The first cell separation unit 300 can also install an automatic scraping device, and when the skin tissue needs to be scraped, the automatic scraping device starts a working state for scraping. The incline plane 320 can facilitate the outflow of the skin tissue residue into the second cell separation unit.

On this basis, when the first cell separation unit 300 is the rectangular tray, diagonals disposed on two sides of the first cell separation unit 300 are lower than the upper surface of the operation plane 310, a thickness of the first cell separation unit 300 is about 1 centimeter (cm), a slope of the incline plane 320 is 10 degrees (°) to 20°, and a length of the incline plane is in a range of 5 cm to 6 cm. The conduit groove 330 can be defined on the incline plane 320 randomly, namely that the conduit groove 330 only needs to be disposed above the opening defined on the second cell separation unit 400. Such a design can concentrate the skin cell suspension scraped by the scalpel in a small area, which is convenient for the collection of skin cell suspension. The conduit groove 330 and the incline plane 320 can enable the scraped skin cell suspension to flow directly into the second cell separation unit 400 without lifting up the first cell separation unit 300 to pour the skin cells into the second cell separation unit 400, so that the operation is simpler and more convenient.

In an illustrated embodiment, since the cell separation operation table includes the operation plane 310 and the incline plane 320 extending outwards from the operation plane 310, when the digested skin tissue is the large patch, the digested skin tissue can be placed on the operation plane 310, and then the cell scraping device such as the scalpel is used to scrape and separate the large-patch digested skin tissue to obtain the first skin cell suspension and the skin tissue residue. Meanwhile, the skin tissue residue may be transferred into the second cell separation unit 400 through the conduit groove 330. Thereafter, the second cell separation unit 400 continues to separate the skin tissue residue, so that the second skin cell suspension can be separated by using the skin tissue residue. Therefore, the skin cells are separated more completely

In an illustrated embodiment, the second cell separation unit 400 of the embodiment of the present disclosure includes: a preparation container 440, a filter screen 420 and a grinding rod 430. Specially, a diameter of an upper opening of the filter screen 420 is greater than a diameter of an opening of the preparation container 440, the filter screen 420 is suspended in the preparation container 440, a mesh diameter of the filter screen is in a range of 100 to 120 meshes, and the grinding rod 430 is used for grinding the skin tissue residue on the filter screen 420.

In an illustrated embodiment, when the second cell separation unit 400 performs cell separation, the to-be-separated skin cells (i.e., the skin tissue residue) can be placed on the filter screen and then gently ground by the grinding rod, the skin cells are separated by the gentle pressure from the rod and the friction between the rod and the mesh, so that more skin cells can be separated.

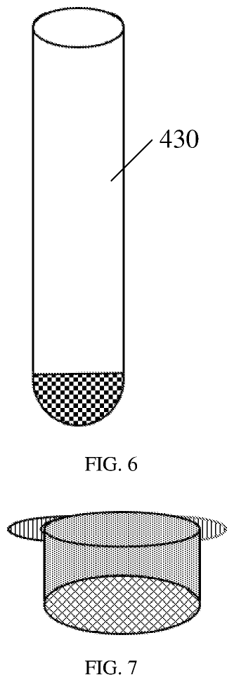

In an embodiment, FIG. 5 is a schematic structural diagram of the filter screen according to an embodiment of the present disclosure, and FIG. 6 is a schematic structural diagram of the grinding rod according to the embodiment of the present disclosure. As shown in FIG. 5 and FIG. 6, the upper opening of the filter screen is circular, a bottom of the filter screen and a bottom of the grinding rod are semicircular, and the bottom of the grinding rod has small protrusions or patterns.

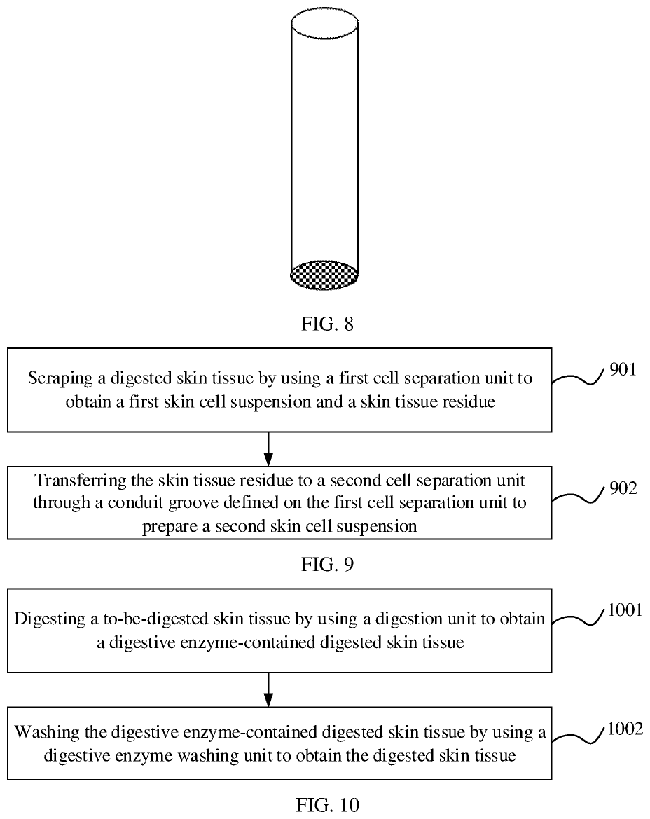

In another embodiment, FIG. 7 is a schematic structural diagram of the filter screen according to another embodiment of the present disclosure, and FIG. 8 is a schematic structural diagram of the grinding rod according to another embodiment of the present disclosure. As shown in FIG. 7 and FIG. 8, the bottoms of the filter screen and the grinding rod are flat, and the bottom of the grinding rod has small protrusions or patterns.

In an illustrated embodiment, the preparation device for the ASCS according to the embodiment of the present disclosure further includes a display screen 500, which is configured to show the temperature of the digestion unit 100 and the time of digestion reacted in the digestion unit 100. Moreover, the preparation device for the ASCS according to the embodiment of the present disclosure further includes a functional panel 600, and the functional panel 600 contains a heating assembly connected to the digestion unit 100 and a circuit control assembly electrically connected to the heating assembly therein. The display screen 500 and the functional panel 600 are further electrically connected to an assembly of batteries and a microchip disposed on the base of the preparation device for the ASCS, which are used to supply and control power for heating the digestion unit 100, the display screen 500 and the functional panel 600.

In an illustrated embodiment, the preparation device for the ASCS of the embodiment of the present disclosure further includes the base, and the base is further provided with a digestion unit base, a digestive enzyme washing unit base and a second cell separation unit base. The embodiment of the present disclosure further includes the functional panel 600 fixed on the base of the preparation device for the ASCS, and the heating assembly and the circuit control assembly in the functional panel 600 are also fixed on the base. The display screen 500 is used for heating display, and the functional panel 600 includes a switch button, a heating button, and a timer button. Specifically, the switch button is used to turn on to supply the power, the heating button is used to heat the digestion unit 100, and the timer button is used to record the digestion time. Therefore, it makes sure not only appropriate time but also appropriate temperature for the digestion, thereby avoiding the damage caused by over-digestion of the skin tissue. Moreover, the functional panel 600 and the display screen 500 are connected with the assembly of batteries and the microchip that fixed on the base.

In an illustrated embodiment, a cavity is defined between the first cell separation unit 300 and the base disposed below the first cell separation unit 300. When the grinding rod 430 is not in use, it can be placed in the cavity defined between the first cell separation unit 300 and the base.

In an illustrated embodiment, the opening 410 defined on the second cell separation unit 400 of the embodiment of the present disclosure is lower than an upper surface of the functional panel 600, and the opening 410 defined on the second cell separation unit 400 is lower than the openings defined on the digestion unit 100 and the digestive enzyme washing unit 200. Therefore, in the process of transferring the skin tissue residue, it is more convenient to transfer the skin tissue residue into the second cell separation unit 400.

In an embodiment, when the digested skin tissue is the large patch, FIG. 9 shows the method for preparing the ASCS provided by the embodiment of the present disclosure, and the method for preparing the ASCS provided by the present disclosure includes the following steps.

Step 901: the first cell separation unit is used to scrape off a digested skin tissue to obtain a first skin cell suspension and a skin tissue residue.

In an illustrated embodiment, when the digested skin tissue is the large patch, the first cell separation unit 300 is first used to scrape the digested skin tissue to obtain the first skin cell suspension and the skin tissue residue, and at this time, the skin tissue residue contains a lot of small patches and un-separated skin cells.

Step 902: the skin tissue residue is transferred to the second cell separation unit for preparation by using the conduit groove defined on the first cell separation unit to obtain a second skin cell suspension.

In an illustrated embodiment, since the first cell separation unit 300 defines the conduit groove 330, the conduit groove 330 is disposed above the opening 410 of the second cell separation unit 400. Therefore, the skin tissue residue can be transferred to the second cell separation unit 400 by using the conduit groove 330 defined on the first cell separation unit 300 for preparation to obtain the second skin cell suspension. Namely, the digested skin tissue is separated by using the first cell separation unit 300 to obtain the first skin cell suspension, and then the skin tissue residue is transferred to the second cell separation unit 400 for secondary separation, so that the skin cells can be separated more completely to obtain the large amount of skin cell suspension.

In another embodiment, when the digested skin tissue is the small patch, the method for preparing the ASCS of the other embodiment of the present disclosure includes the following steps: preparing the digested skin tissue by using the second cell separation unit 400 to obtain a third skin cell suspension.

In an illustrated embodiment, when the digested skin tissue is the small patch, the second cell separation unit 400 is directly used to separate the skin cell suspension (i.e., the third skin cell suspension). Therefore, no matter whether the digested skin tissue is the large patch or the small patch, the digested skin tissue can be prepared through the first cell separation unit 300 and the second cell separation unit 400, so that the skin cells are more completely separated, and the separation efficiency of the skin cells is improved.

For example, the method for preparing the ASCS according to the embodiment of the present disclosure further includes: scraping the skin tissue residue into the conduit groove defined on the incline plane, and transferring the skin tissue residue to the second cell separation unit by using the conduit groove.

In an illustrated embodiment, since the cell separation operation table includes the operation plane 310 and the incline plane 320 extending outwards from the operation plane 310, when the digested skin tissue is the large patch, the digested skin tissue is placed on the operation plane 310, and then the cell scraping device such as the scalpel is used to scrape and separate the large-patch digested skin tissue to obtain the first skin cell suspension and the skin tissue residue. At this time, the skin tissue residue is transferred into the second cell separation unit 400 through the conduit groove. Thereafter, the second cell separation unit 400 continues to separate the skin tissue residue, so that the second skin cell suspension is further separated by using the skin tissue residue. Therefore, the skin cells are separated more completely, and a large amount of the skin cell suspension can be obtained.

In an illustrated embodiment, FIG. 10 shows a flowchart for preparing a to-be-digested skin tissue according to an embodiment of the present disclosure. As shown in FIG. 10, before the first cell separation unit is used to scrape off the digested skin tissue, the method for preparing the ASCS according to the embodiment of the present disclosure further includes the following steps.

Step 1001: the to-be-digested skin tissue is digested by using the digestion unit to obtain a digestive enzyme-contained digested skin tissue. The digestion unit includes a digestive enzyme.

Specially, trypsin is selected for the digestion, the trypsin is the most widely used digestion reagent at present, and the trypsin acts on a peptide bond linked to lysine or arginine to remove intercellular mucin and glycoprotein, thereby separating the skin cells. In the present disclosure, 2.5 milligrams (mg) of trypsin is dissolved with 10 milliliters (mL) of sterile water, the trypsin and the sterile water are uniformly mixed and then injected into the digestion tank, and the button for heating is pressed. Thereafter, the to-be-digested skin tissue is added when the temperature reaches 37 degrees Celsius (° C.), the button for timing is pressed for 20-25 minutes, and then the skin tissue is taken out.

Step 1002: the digestive enzyme-contained digested skin tissue is washed by using the digestive enzyme washing unit to obtain the digested skin tissue. Specially, the digestive enzyme washing unit includes digestive enzyme termination solution therein.

In an illustrated embodiment, the digestive enzyme-contained digested skin tissue is put into the digestive enzyme washing unit for repeated washing, so that the digestive enzyme remained on the digestive enzyme-contained digested skin tissue is reduced, and the digestion can be stopped to obtain the digested skin tissue.

In an illustrated embodiment, the digested skin tissue of the embodiment of the present disclosure can be prepared into the small patch, and then the skin cells are separated according to the above method. In addition, the skin cells are scraped by the scalpel on the cell separation operation table firstly, then the skin cells mixture are poured into the filter screen to be filtered, and then the unfiltered skin cell masses and the skin tissue residue are separated again by using the grinding rod, and finally, a buffer solution is added into the suspension tank to the required volume.

In an illustrated embodiment, the method for preparing the ASCS according to the embodiment of the present disclosure further includes: heating the digestion unit by using the heating assembly, and controlling the heating time and the heating temperature of the heating assembly by using the circuit control assembly, thereby providing suitable digestive conditions for the digestion unit. The digestive conditions include: 20 min to 25 min of digestion time and 37° C. of digestion temperature. Under the digestive conditions, the to-be-digested skin tissue can be digested more fully to prepare for the next separation.

It can be seen from the above that the present disclosure provides the preparation device for the ASCS, which is provided with the digestion tank, the digestive enzyme washing tank, the suspension tank, the heating timing switch, the temperature display and the time display, the filter screen for grinding the skin cells, the grinding rod, the cell separation operation table and accessories, etc. The preparation device for the ASCS can directly uses the grinding rod to grind the skin tissue on the filter screen, and the digested skin tissue treated with the digestive enzyme is gently ground and rubbed to separate the skin cells. In this situation, the pressure generated by the grinding rod interacts with a shear force formed between meshes of the filter screen and the grinding rod, which is equivalent to a matrix effect of countless dense blades. Therefore, the speed of scraping the skin cells in the present disclosure is greatly improved compared to using a single blade of the regular scalpel. Moreover, the large number of cell aggregates and residual tissue fragments formed in scraping the skin cells with the single blade of the regular scalpel are avoided. Therefore, the separation efficiency of skin cells is also greatly improved. Compared with a traditional surgical scalpel scraping and separation method, the method provided by the present disclosure is more convenient, faster and more complete in separating the skin cells. Namely, the present disclosure can separate more skin cells and has the higher separation efficiency. The method provided by the present disclosure can be used alone or combined with the traditional method to improve the separation efficiency of the skin cells.

### Embodiment 1

2.5 mg of trypsin is dissolved in 10 mL of sterile water, the trypsin and the sterile water are uniformly mixed and then injected into a digestion tank, the button for heating is pressed, and the to-be-digested skin tissue is added when the temperature of the digestion tank reaches at 37° C. The button for timing is pressed to prepare the digestive enzyme-contained digested skin tissue, after 25 min, the digestive enzyme-contained digested skin tissue is taken out and washed repeatedly in a washing tank to obtain the digested skin tissue, and then the digested skin tissue prepared into the small piece is directly placed in a filter screen on the suspension tank, and the skin cells are separated by gentle pressurization and friction with the grinding rod. In addition, the digested skin tissue prepared into the large piece is cut by a scissor into small patches, the skin cells are separated according to the above method, and finally, a buffer solution is added into the suspension tank to a required volume.

### Embodiment 2

2.5 mg of trypsin is dissolved in 10 mL of sterile water, the trypsin and the sterile water are uniformly mixed and then injected into a digestion tank, the button for heating is pressed, and the to-be-digested skin tissue is added when a temperature of the digestion tank reaches at 37° C. The button for timing is pressed to prepare the digestive enzyme-contained digested skin tissue, after 25 min, the digestive enzyme-contained digested skin tissue is taken out and washed repeatedly in a washing tank to obtain the digested skin tissue, and then the digested skin tissue prepared into the large piece is first scraped to obtain the skin cells on the cell separation operation table by using the scalpel; then, the scalped digested skin tissue mixture is poured into the filter screen for filtering; the unfiltered skin cells aggregates and tissue residue are further separated by the grinding rod again; and finally, a buffer solution is added into the suspension tank to a required volume.

### Comparative Example 1

A traditional mechanical separation method is used to prepare the ASCS in the comparative example 1.

### Comparative Example 2

The ASCS is prepared by using a scalpel scraping digestive enzyme separation method in the comparative example 2.

Effects of the embodiments of the method for preparing the ASCS of the present disclosure are compared with effects of the comparative examples as follows.

| | Embodiment | Embodiment | Comparative | Comparative |
|---|---|---|---|---|
| | 1 | 2 | example 1 | example 2 |
| Separation | 85-90 | 85-90 | 30-40 | 70-80 |
| efficiency of | | | | |
| skin cells | | | | |

It can be seen from the above table that the separation efficiencies of the skin cells separated by the preparation device for the ASCS according to the embodiments of the present disclosure are far greater than the comparative examples 1-2. Therefore, the preparation device for the ASCS provided by the embodiments of the present disclosure can improve the separation efficiency of the skin cells.

Although the present disclosure has been described in conjunction with specific features and embodiments thereof, it will be apparent that various modifications and combinations may be made thereto without departing from the spirit and scope of the present disclosure. Accordingly, the present disclosure and the attached drawings are merely illustrative as defined by the present disclosure, and are to be considered as covering any and all modifications, variations, combinations or equivalents within the scope of the present disclosure. Apparently, those skilled in the related art can make various modifications and variations to the present disclosure without departing from the spirit and scope of the present disclosure. Thus, if these modifications and variations of the present disclosure fall within the scope of the present disclosure and their equivalent technologies, the present disclosure is also intended to include these modifications and variations.

The above description is only the illustrated embodiments of the present disclosure, but the scope of the protection of the present disclosure is not limited thereto. Furthermore, any changes or substitutions can be easily conceived of by those skilled in the related art within the technical scope disclosed by the present disclosure, which should be covered by the scope of the protection of the present disclosure. Therefore, the scope of the protection of the present disclosure shall be subject to the description of the present disclosure.

What is claimed is:

1. A method for preparing autologous skin cell suspension (ASCS), wherein the method for preparing the ASCS is applied in a preparation device for the ASCS; the preparation device for the ASCS comprises: a digestion unit, a digestive enzyme washing unit, a first cell separation unit, and a second cell separation unit; and the first cell separation unit defines a conduit groove, and the conduit groove is disposed above the second cell separation unit; and
wherein the method for preparing the ASCS comprises at least one of the following steps:
scraping a digested skin tissue by using the first cell separation unit to obtain a first skin cell suspension and a skin tissue residue; and transferring the skin tissue residue to the second cell separation unit through the conduit groove defined on the first cell separation unit to prepare a second skin cell suspension; and
preparing the digested skin tissue by using the second cell separation unit to obtain a third skin cell suspension.

2. The method for preparing the ASCS as claimed in claim 1, wherein the first cell separation unit further comprises: a cell separation operation table, and the scraping a digested skin tissue by using the first cell separation unit to obtain a first skin cell suspension and a skin tissue residue specially comprises:
scraping the digested skin tissue by using the cell separation operation table to obtain the first skin cell suspension and the skin tissue residue.

3. The method for preparing the ASCS as claimed in claim 2, wherein the cell separation operation table comprises: an operation plane and an incline plane extending outwards from the operation plane; the conduit groove is defined on the incline plane, the first cell separation unit is connected to an opening defined on the second cell separation unit through the conduit groove; and
wherein the transferring the skin tissue residue to the second cell separation unit through the conduit groove defined on the first cell separation unit specially comprises:
transferring the skin tissue residue to the second cell separation unit through the conduit groove.

4. The method for preparing the ASCS as claimed in claim 1, wherein before the scraping a digested skin tissue by using the first cell separation unit, the method for preparing the ASCS further comprises the following steps:
digesting a to-be-digested skin tissue by using the digestion unit to obtain a digestive enzyme-contained digested skin tissue, wherein the digestion unit comprises a digestive enzyme therein;
washing the digestive enzyme-contained digested skin tissue by using the digestive enzyme washing unit to obtain the digested skin tissue, wherein the digestive enzyme washing unit comprises a digestive enzyme termination solution therein.

5. The method for preparing the ASCS as claimed in claim 1, wherein the second cell separation unit comprises: a preparation container, a filter screen, and a grinding rod;
wherein the transferring the skin tissue residue to the second cell separation unit through the conduit groove defined on the first cell separation unit to prepare a second skin cell suspension specially comprises:
placing the skin tissue residue onto the filter screen; and
grinding the skin tissue residue by using the grinding rod to obtain the second skin cell suspension.

6. The method for preparing the ASCS as claimed in claim 1, wherein the second cell separation unit comprises: a preparation container, a filter screen, and a grinding rod;
wherein the preparing the digested skin tissue by using the second cell separation unit to obtain a third skin cell suspension specially comprises:
placing the digested skin tissue onto the filter screen; and
grinding the digested skin tissue by using the grinding rod to obtain the third skin cell suspension.

7. The method for preparing the ASCS as claimed in claim 1, wherein the preparation device for the ASCS further comprises: a heating assembly connected to the digestion unit, and the method for preparing the ASCS further comprises:
heating the digestion unit by using the heating assembly.

8. The method for preparing the ASCS as claimed in claim 7, wherein the preparation device for the ASCS further comprises: a circuit control assembly electrically connected to the heating assembly, and the method for preparing the ASCS further comprises:
controlling a heating time and a heating temperature of the heating assembly by using the circuit control assembly to provide digestive conditions for the digestion unit.

9. The method for preparing the ASCS as claimed in claim 8, wherein the digestive conditions of the digestion unit comprise: 20 minutes (min) to 25 min of digestion time and 37 degrees Celsius (° C.) of digestion temperature.

10. The method for preparing the ASCS as claimed in claim 1, wherein an opening defined on the second cell separation unit is lower than an opening defined on the digestive enzyme washing unit, and the method for preparing the ASCS further comprises:
scraping the digested skin tissue treated by the digestive enzyme washing unit into the second cell separation unit.
